(19) United States
(12) Patent Application Publication
(10) Pub. No.: US 2014/0128810 A1
(43) Pub. Date: May 8, 2014

(54) PERCUTANEOUS MEDICATION DEVICE AND NEEDLE FORMATION USED FOR THE DEVICE

(71) Applicants: Hiroshi Ozawa, Shizuoka (JP); Akinori Inou, Shizuoka (JP); Toru Sekiguchi, Tokyo (JP)

(72) Inventors: Hiroshi Ozawa, Shizuoka (JP); Akinori Inou, Shizuoka (JP); Toru Sekiguchi, Tokyo (JP)

(73) Assignee: NANBU PLASTICS CO., LTD., Shizuoka (JP)

(21) Appl. No.: 14/003,887

(22) PCT Filed: Mar. 21, 2012
(86) PCT No.: PCT/JP2012/001936

§ 371 Date: Dec. 10, 2013

(30) Foreign Application Priority Data
Mar. 23, 2011 (JP) 2011-065080
Nov. 2, 2011 (JP) 2011-241709

(51) Int. Cl. A61M 5/32

(52) U.S. Cl. CPC A61M 5/3298
USPC 604/173

(57) ABSTRACT

A percutaneous medication device according to the present invention comprises: a syringe into which a medical liquid is filled; and a needle formation attached to a tip of the syringe; where: the needle formation comprises a plurality of needles protruding from a surface of a tip of the needle formation; a tip of the needle is cut diagonally with respect to the longitudinal direction of the needle; a discharge opening formed at the tip of each needle is facing substantially opposite from the center direction of the needle formation; and a pitch between the adjacent needles is 1 mm to 10 mm.

### TECHNICAL FIELD

The present invention relates to a percutaneous medication device for percutaneously administering a medical liquid and a needle formation used for the percutaneous medication device.

### BACKGROUND ART

Conventionally, a syringe has been used for administrating a medical liquid or medicine (hereinafter, collectively referred to as medical, liquid) which cannot be perorally administered. However, the level of stress to a body in such a method using a syringe is large and is accompanied by pain. Although a method for percutaneously administering a medicine with a patch has been used, this method takes a long time to express a medical effect and usable medicine is limited.

In order to solve these problems, a percutaneous medication device is proposed which uses a needle with a small diameter to reduce the pain due to piercing by the needle during injection. However, there is a disadvantage of increase in flow resistance during injection of a medical liquid when the diameter of the needle becomes smaller.

Accordingly, Japanese National Phase PCT Laid-Open Publication No. 2005-527249 (Patent Literature 1), Japanese Laid-Open Publication No. 2005-87521 (Patent Literature 2) and Japanese Laid-Open Publication No. 2003-135598 (Patent Literature 3) propose percutaneous medication devices which use a needle with a small diameter and a plurality of the needles.

While these percutaneous medication devices are said to be capable of simultaneously injecting a medical liquid at numerous different points into an affected part, the openings at the tips of the needles are directed randomly. Thus, there is a disadvantage of the medical liquid injected from each needle into an affected area not being injected into a desired part.

### CITATION LIST

### Patent Literature

PTL 1: Japanese National Phase PCT Laid-Open Publication No. 2005-527249

PTL 2: Japanese National Phase PCT Laid-Open Publication No. 2005-87521

PTL 3: Japanese Laid-Open Publication No. 2003-135598

### SUMMARY OF INVENTION

### Technical Problem

The present invention is intended to solve the conventional problems described above. It is an objective of the present invention to provide: a percutaneous medication device capable of injecting a medical liquid into a desired part of the skin while causing no pain during the injection and reducing the flow resistance of the medical liquid; and a needle formation used for the device.

### Solution to Problem

In order to solve the problems described above, the present invention is characterized as follows.

A percutaneous medication device according to the present invention comprises: a syringe into which a medical liquid is filled; and a needle formation attached to a tip of the syringe; where: the needle formation comprises a plurality of needles protruding from a surface of a tip of the needle formation; a tip of the needle is cut diagonally with respect to the longitudinal direction of the needle; a discharge opening formed at the tip of each needle is facing substantially opposite from the center direction of the needle formation; and a pitch between the adjacent needles is 1 mm to 10 mm.

Its the percutaneous medication device according to the present invention, the number of needles protruding from the surface of the tip of the needle formation is 2 to 10.

In the percutaneous medication device according to the present invention, the number of needles protruding from the surface of the tip of the needle formation is three.

In the percutaneous medication device according to the present invention, the protruding dimension of the needle protruding from the surface of She tip of the needle formation is within the range of 0.1 mm to 10 mm.

In the percutaneous medication device according to the present invention, a pitch between the adjacent needles is within the range of 1 mm to 3 mm.

In the percutaneous medication, device according to the present invention: the needle formation comprises a needle formation body, in which the needle protrudes from the surface of the tip thereof, and a cover portion for covering a tip portion of the needle formation body; the needle is inserted into a through-hole formed at the tip portion of the needle formation body and the needle is fixed to the needle formation body by a fixation member; the needle formation body is engaged with the covering portion, and a space is formed between the inner surface of the covering portion and the tip surface of the needle formation body; and the needle protrudes from a surface of a tip of the covering portion, through a needle insertion hole formed at a tip portion of the covering portion.

In a needle formation according to the present invention attached to a tip portion of a syringe: the needle formation comprises a plurality of needles protruding from a surface of a tip of the needle formation; a tip of each of the needles is cut diagonally with respect to the longitudinal direction of the needle; a discharge opening formed at the lip of each of the needles is facing substantially opposite from the center direction of the needle formation; and a pitch between the adjacent needles is within the range of 1 mm to 10 mm.

A percutaneous medication device according to the present invention comprises: a syringe into which a medical liquid is filled; and a needle formation attached to a tip of the syringe; where: the needle formation comprises a plurality of needles protruding from a surface of a tip of the needle formation; a tip of each of the needles is cut diagonally with respect to the longitudinal direction of the needle; a discharge opening formed at the tip of each of the needles is facing substantially the center direction of the needle formation; and a pitch between the adjacent needles is within the range of 1 mm to 10 mm.

In a needle formation according to the present invention attached to a tip portion of a syringe: the needle formation comprises a plurality of needles protruding from a surface of a tip of the needle formation; a tip of each of the needles is cut diagonally with respect to the longitudinal direction of the needle; a discharge opening formed at the tip of each of the needles is facing substantially the center direction of the needle formation; and a pitch between the adjacent needles is within the range of 1 mm to 10 mm.

### Advantageous Effects of Invention

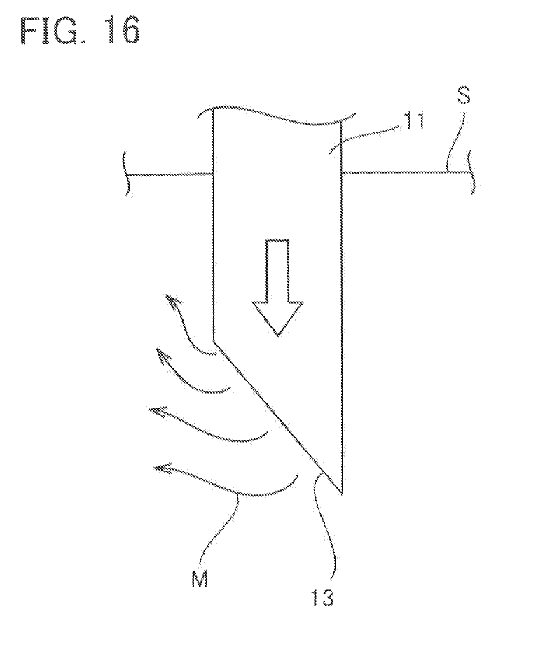

The needle formation used in the percutaneous medication device according to the present invention comprises a plurality of needles, the tip of each of the needles is cut diagonally with respect to the longitudinal direction of the needle, and the discharge opening formed at the tip of each needle is facing substantially opposite from the center direction of the needle formation, so that a medical liquid M will be injected into the skin from the discharge opening of each needle as shown by the arrows in FIG. 16. Further, since the pitch between the plurality of needles is within the range of 1 mm to 10 mm, the medical liquid is supplied almost equally in the periphery of the piecing positions of the respective needles, and at a desired depth, as a whole. As a result, the medical liquid can be effectively injected into, for example, the epidermal layer, the derm layer, or the subcutaneous layer of the skin, and the dose of the medical liquid can be reduced.

The needle formation is configured of a needle formation body, in which a needle protrudes from the surface of the tip thereof, and a covering portion for covering the tip portion of the body, and a space is formed in between the inner surface of the covering portion and the tip surface of the needle formation body. Thus, the fixation member for fixing the needle to the needle formation body is arranged within this space, and the variation in the applied amount (size of the solidified matter) of the fixation member will not influence the protruding dimension of the needle from the surface of the covering portion.

Further, even in a case where the discharge opening formed at the tip of each needle is configured to face the center direction of the needle formation, the medical liquid M is injected into the skin from the discharge opening of each needle as shown by the arrows in FIG. 16. Further, since the pitch between the plurality of needles is within the range of 1 mm to 10 mm, the medical liquid is supplied almost equally in the periphery of the piecing positions of the respective needles, and at a desired depth, as a whole. As a result, the medical liquid can be effectively injected into, for example, the epidermal layer, the derm layer, or the subcutaneous layer of the skin, and the dose of the medical liquid can be reduced.

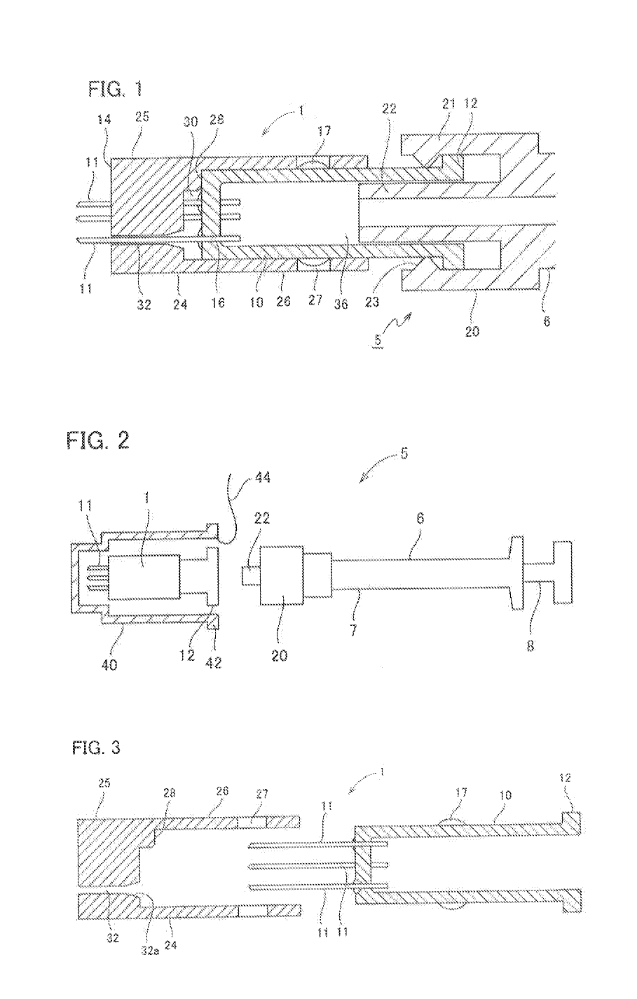

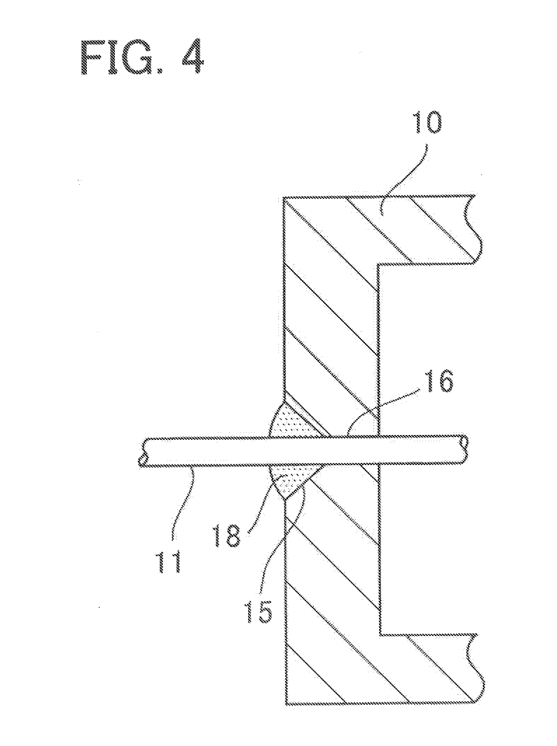

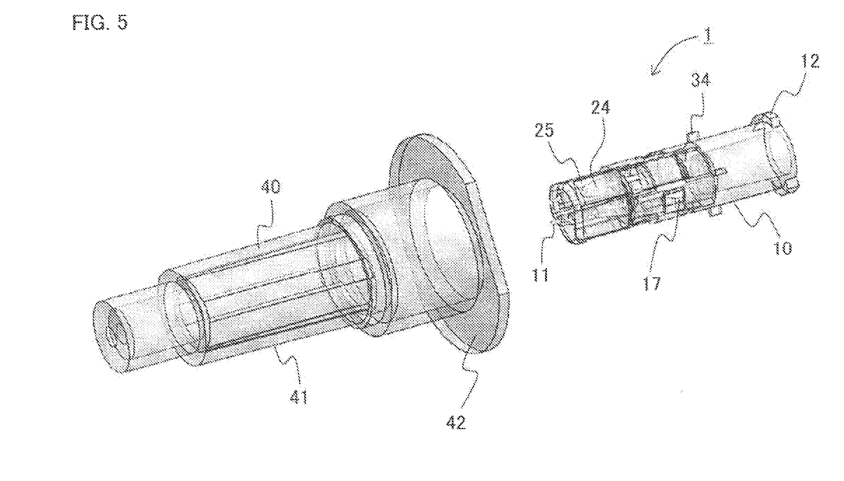

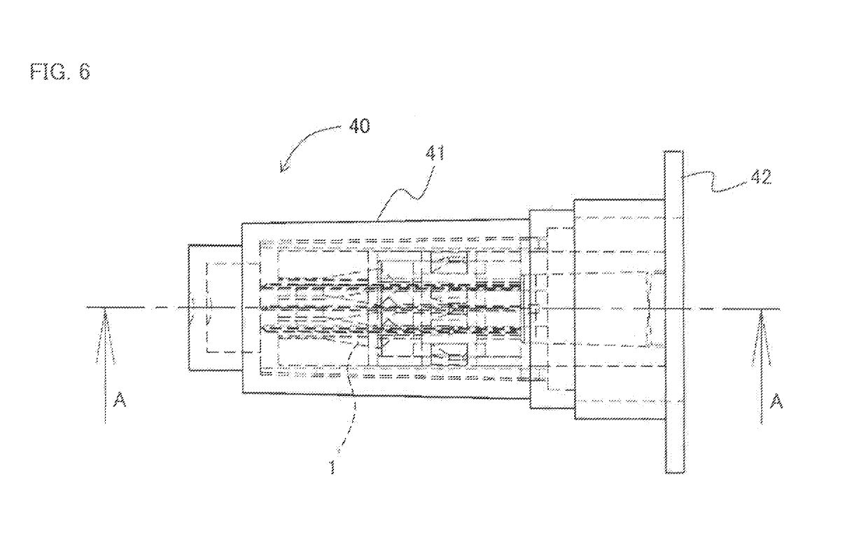

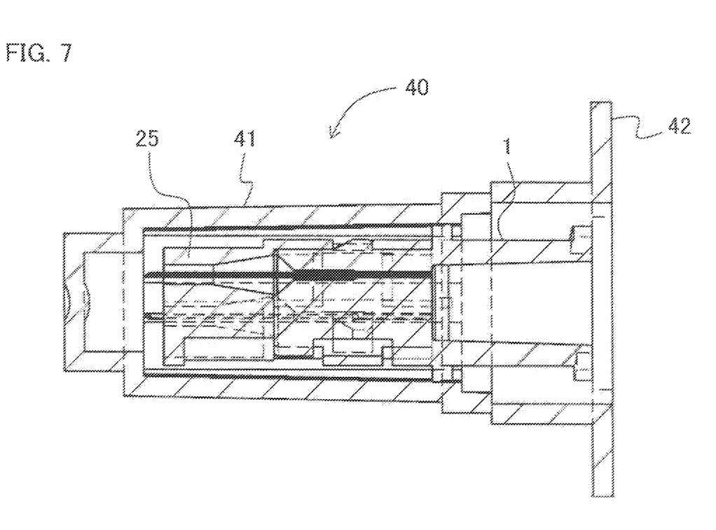

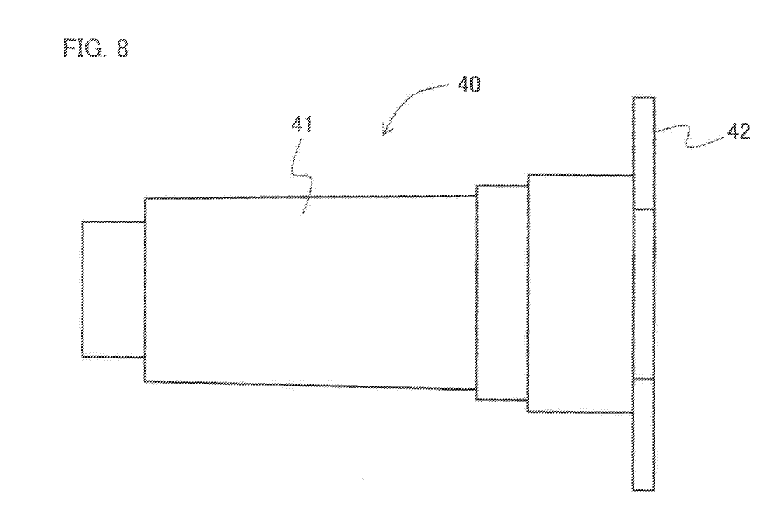

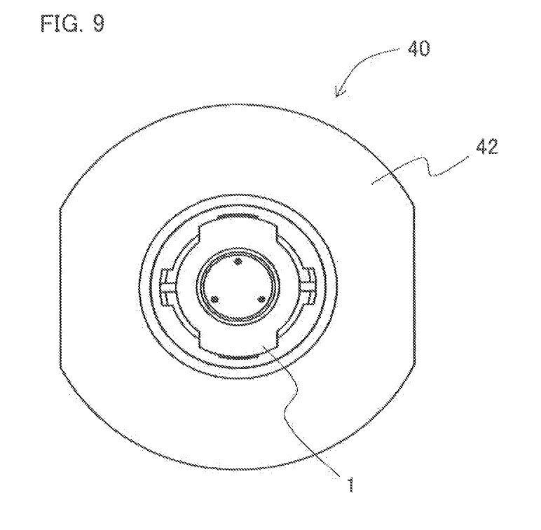

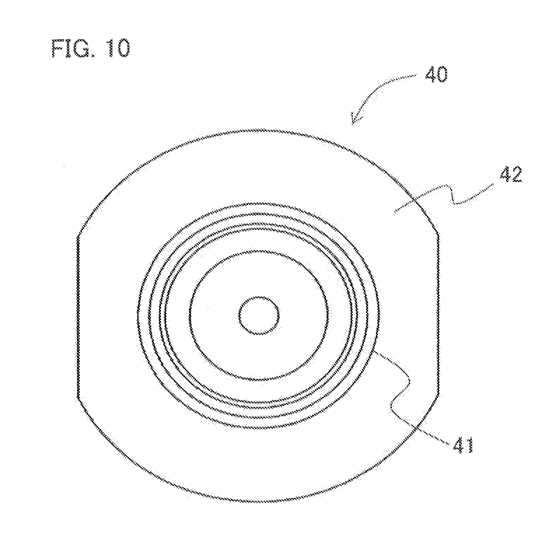

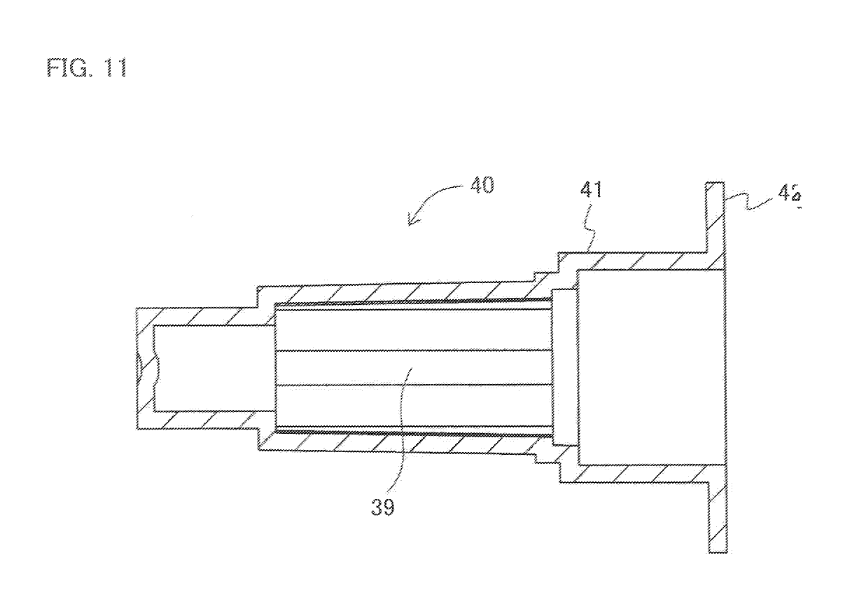

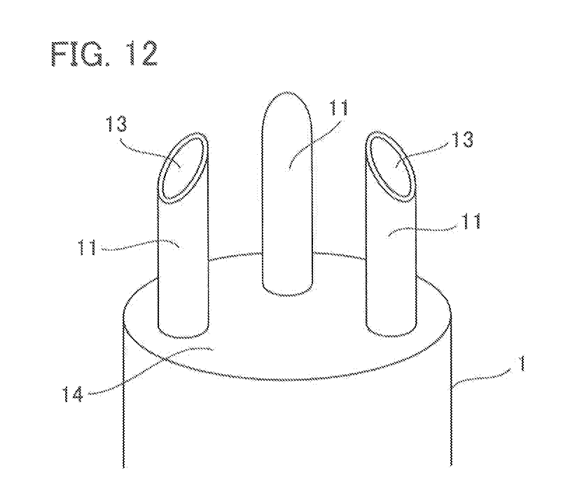

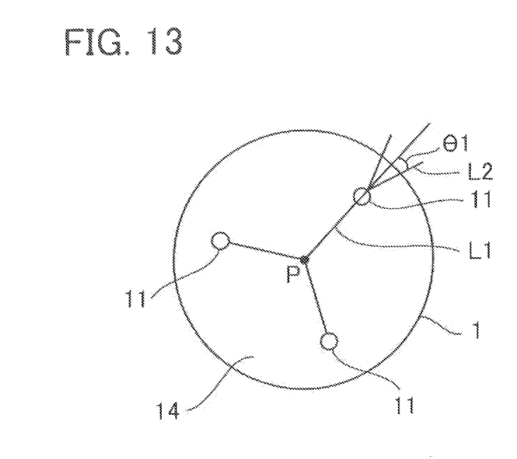

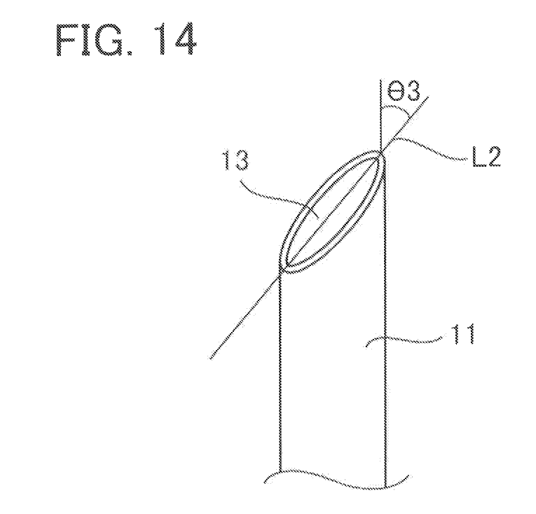

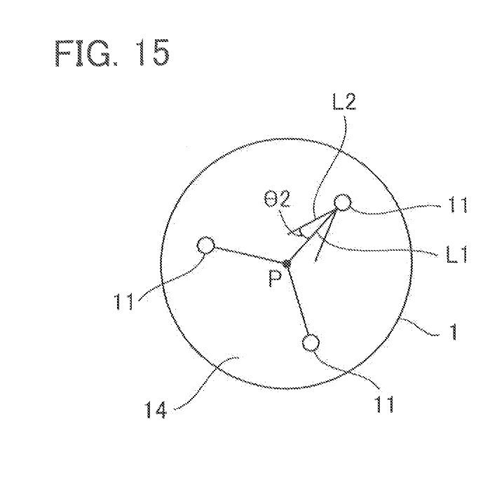

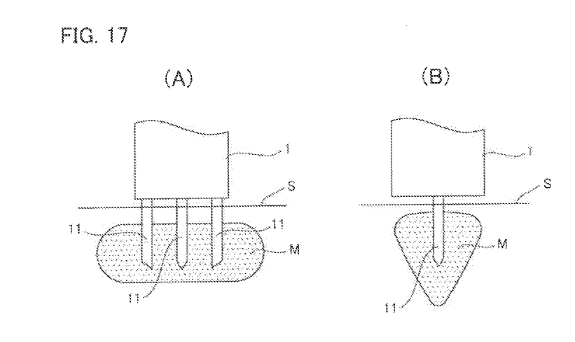

### BRIEF DESCRIPTION OF DRAWINGS

[FIG. 1] FIG. 1 is a cross sectional view, where a needle formation is attached to a tip of a syringe.

[FIG. 2] FIG. 2 is a cross sectional view of an essential part, where a needle formation is arranged within a needle holder.

[FIG. 3] FIG. 3 is an exploded cross sectional view of a needle formation body and a covering portion, which constitutes a needle formation.

[FIG. 4] FIG. 4 is an enlarged cross sectional view of an essential part, of a needle formation.

[FIG. 5] FIG. 5 is a perspective view of a needle formation and a needle holder.

[FIG. 6] FIG. 6 is an elevation view, where the needle formation shown in FIG. 5 is housed within a needle holder.

[FIG. 7] FIG. 7 is a cross sectional view along the line A-A,

[FIG. 8] FIG. 8 is an elevation view of FIG. 6.

[FIG. 9] FIG. 9 is a side view from the open side of FIG. 6.

[FIG. 10] FIG. 10 is a side view from the closed side of FIG. 6.

[FIG. 11] FIG. 11 is a cross sectional view of a needle holder.

[FIG. 12] FIG. 12 is a perspective view of a tip portion of a needle formation.

[FIG. 13] FIG. 13 is an explanatory diagram showing an arrangement of needles and a direction of discharge openings of a needle, formation.

[FIG. 14] FIG. 14 is a perspective view of a discharge opening portion formed at a tip of a needle.

[FIG. 15] FIG. 15 is an explanatory diagram showing an arrangement of needles and a direction of discharge openings of a needle formation in another embodiment.

[FIG. 16] FIG. 16 is a diagram describing the flow of a medical liquid from a needle tip.

[FIG. 17] FIG. 17 is an explanatory diagram showing a distribution of a medical liquid injected from a needle tip into the skin.

### DESCRIPTION OF EMBODIMENTS

Hereinafter, an embodiment of the present invention will be described in detail with reference to accompanying figures.

As shown in FIGS. 1 to 11, a percutaneous medication device 5 of the present invention comprises: a syringe 6, in which a medical liquid is filled; a needle formation 1 attached to a tip of the syringe 5.

The syringe 6 comprises: a syringe, body 7; a plunger 8, which is inserted into the syringe body 7; and a Luer lock portion 20 formed at the tip of the syringe body 7. The Luer lock portion 20 comprises an outer cylinder 21 and an inner cylinder 22, where a screw 23 is formed on an inner surface of the outer cylinder 21.

The needle formation 1 comprises: a needle formation body 10 in which a needle 11 projects from the tip surface thereof; and a cover portion 24 covering the tip of the needle, formation body 10. The needle formation body 10 is formed in a cylindrical shape, one end of which is open. A flange 12 which can engage the Luer lock portion 20 is formed on a base end of the needle formation body 10. At the tip of the needle formation body 10, a through-hole 16 is formed to allow the needle 11 to pass therethrough. As shown in FIG. 12, in the present embodiment, three through-holes 16 are formed; however, the through-holes formed may be two or, four or more. The number of through-holes is preferably 2 to 10, and more preferably 3 to 6.

The needle 11 is inserted into the through-hole 16 and the needle 11 is fixed to the needle formation body 10 by a fixation member 18 such as adhesive. The fixation method of the needle 11 is not limited to methods which use adhesive; for example, methods such as fusion (heat fusion, ultrasonic fusion, high frequency fusion) and the like may be used.

As shown in FIG. 4, a mortar-shaped recess 15 is formed on a surface of a tip of the needle formation body 10, in the periphery of the through-hole 16, and a fluid adhesive 18 can be applied into the through-hole 16 through the recess 15.

On an outer surface of the needle formation body 10, a semi-circular protrusion 17 is projected as an engaging portion.

The cover portion 24 comprises: a head portion 25; and a plurality of resilient pieces 26 which are extended to the side closer to the needle formation body 10 from the head portion 25. In the head portion 25, a needle insertion hole 32, through which the needle 11 can pass, is formed at the position corresponding to the attachment position of the needle 11. Three resilient pieces 26 are integratedly formed from the head portion 25 in the present embodiment; however, the resilient pieces 28 formed may be two or four or more.

In the resilient pieces 26, an engaging hole 27 is formed as an engaged portion at the position corresponding to an engaging portion 17 formed on an outer surface of the needle formation body 10.

On an inner surface of the head portion 25, a step portion 28 is formed. When the needle formation body 10 is inserted into and mated with an inside of the cover portion 24, the tip surface of the needle formation body 10 abuts against the step portion 28 to form a space 30 in between an inner surface of the head portion 25 of the cover portion 24 and the tip surface of the needle formation body 10 (FIG. 1). At the same time, the engaging portion 17 of the needle formation body 10 engages in a snap manner with the engaged portion 27 formed in the resilient piece 26 of the cover portion 24.

Thus, when the needle formation body 10 mates with the cover portion 24, the needle 11 projects from the tip surface of the cover portion 24 through the needle insertion hole 32 formed at the tip of the cover 24.

As shown in FIG. 3, the inside diameter of the needle insertion hole 32 is formed slightly larger than the outer diameter of the needle 11. Additionally, the needle insertion hole 32 is formed in a cone so that the inner diameter becomes larger towards the side closer to the needle formation body 10. By forming a conical shaped portion 32a with a tapered shape at an open side of the needle insertion hole 32, the needle 11, which projects from, the tip of the needle formation body 10, can be inserted into the insertion hole 32 easily and without a resin of the cover portion 24 adhering to a needle tip.

Examples of the constitution material for the needle 11 include, but not limited to, a metallic material such as stainless steel, aluminum or aluminum alloy, or titanium or titanium alloy, plastic material, or the like. The projection dimension of the needle 11 projecting from the surface of the needle formation 1 is preferably equal to or less than 10 mm; and the projection dimension can be 5 mm or less, and in particular, 3 mm or less. Furthermore, the projection dimension of the needle 11 projecting from the surface of the needle formation 1 is more preferably within the range of 0.5 mm to 2.0 mm, and still more preferably, within the range of 0.5 mm to 1.5 mm. The maximum outer diameter of the needle 11 is more preferably within the range of 0.1 mm to 0.6 mm, and, still more preferably, within the range of 0.1 mm to 0.4 mm. The tip of the needle 11 has a shape that appears to be an obliquely-cut tubular member (FIG. 12). The angle theta3 of the cut surface of the tip of the needle 11 is preferably 10 to 50 degrees with respect to the longitudinal direction of the needle 11 (FIG. 14). Typically, the diagonal cut surface formed at the tip of the needle 11 is cut diagonally with respect to the longitudinal direction of the needle, and the middle point of this first cut surface is further cut diagonally to form a second cut surface.

In the present embodiment, while the number of the plurality of needles 11 attached to the surface portion of the needle formation 1 is three, the number of the needles may be 2 or 4 or more. The number is preferably 2 to 10, and more preferably 3 to 6. The plurality of needles 11 are separated at equal intervals from the center point of the surface of the needle formation 1, and the angles (internal circumference angle) between, the plurality of needles 11 around the center point are set to be the same angle with one another. For example, when the outer diameter of the surface portion of the needle formation 1 is 10 mm to 20 mm and three needles 11 are provided, the three needles 11 are arranged 1 mm to 1.25 mm (preferably 1 mm to 1.5 mm) away from the center point on a surface portion 14, and the angles (internal circumference angle) between the plurality of needles 11 around the center point are set to be 120 degrees. The pitch (distance) between adjacent needles 11 is preferably 1 mm to 5 mm, or 5 mm to 10 mm, and more preferably 1 mm to 5 mm, and still more preferably 1 mm to 3 mm. In particular, respective needle 11 are preferably arranged on a circle around the center point and at equal intervals. In this case, the respective needles are preferably arranged on a circle with a PCD (Pitch Circle Diameter) of 2 mm to 5 mm, and more preferably on a circle with a PCD of 2 mm to 3 mm. The respective needles 11 may also be arranged with different intervals on a circle around the center point. Further, the plurality of needles 11 may be arranged with different intervals from the center point of the surface of the needle formation 1.

As shown in FIG. 12, the direction of a discharge opening 13 formed at the tip of the needle 11 is, in the present embodiment, in a direction substantially opposite from the center direction of the needle formation (also referred to as substantially outwards) with regard to a plurality of needles 11. In the present invention, the discharge opening 13 facing substantially the opposite direction from the center direction of the needle formation 1 means that the discharge opening 13 is facing opposite from the center direction of the needle formation 1 and, from the view point of the axial direction, the deviation angle theta1 of a segment L2 connecting the upper end edge and the lower end edge of the discharge opening 13 is within 15 degrees with respect to a segment L1 connecting the central axis of the needle 11 and the center (P) of the needle formation 1 (FIGS. 13 and 14).

When the number of the needles 11 is two, the respective discharge openings 13 of the needles 11 face opposite from the center direction of the needle formation 1. When the number of the needles 11 is three, the respective discharge openings 13 of the needles 11 face opposite from the center direction of the needle formation 1.

As such, by adjusting the respective discharge openings 13 of a plurality of the needles 11 to face substantially outwards, a medical liquid sent from each needle 11 into tissues of the skin can be sent to a desired portion.

Specifically, as shown in FIG. 16, the medical liquid discharged from the discharge opening 13 of the needle 11 tends to flow in the direction orthogonal to the opening surface of the discharge opening 13. Since the respective discharge openings 13 of the plurality of adjacent needles 11 are facing outwards, the flow of the medical liquid is almost in a circular shape as a whole from the view point on the part of the skin, and the medical liquid M is injected into the skin S at a predetermined depth from the surface of the skin (FIG. 17A). Specifically, when the medical liquid is discharged into cutaneous tissues from the discharge opening 13 of the needle 11, the medical liquid can be injected at a desired portion of cutaneous tissues (the epidermis, the derm, and the like) in a dispersed state, without the needle 11 or needle formation 1 moving. Compared to the pushing pressure of the medical liquid within the needle formation 1, the pressure of the medical liquid discharged from each needle 11 into the tissues is smaller (in a case of three needles, the discharge pressure of the medical liquid from each needle is about ⅓). Thus, the medical liquid is thought to be dispersed within the tissues, without going into a deeper portion.

On the contrary, when one needle is used to inject a medical liquid into the skin S, the medical liquid M is injected into the inside of the skin S as shown in FIG. 17B, and the dispersion of the medical liquid M is poor. Since the pushing pressure of the medical liquid within the needle formation 1 is maintained and the medical liquid is discharged from one needle 11 into the tissues, it is thought that the medical liquid goes into the deeper portion of the tissues and the medical liquid does not disperse.

The needle formation 1 can be manufactured by injection molding and the like with a thermoplastic resin, such as polycarbonate, polypropylene, ABS resin, polystyrene and the like, as a material.

A needle holder 40 comprises, as shown in FIGS. 5 to 11, a holder body 41 formed in a generally cylindrical shape, one end of which is open, and a flange 42 formed at the open end portion side of the holder body 41. A recess groove (spline) 39 is formed running in the axial direction in the inner surface of the holder body 41 (FIG. 11). A protrusion section 34 is formed on an outer surface of the needle formation 1 in such a manner to engage with the recess groove 39. When the needle formation 1 is inserted into the holder body 41, the protrusion section 34 engages with the recess groove 39 that is formed in the inner surface of the holder body 41, so that the needle formation 1 cannot be rotated relative to the holder body 41.

A seal member 44, which can close the open end portion of the holder body 41, is adhered to the flange 42 of the holder body 41. The seal member 44 seals the open end of the holder body 41 to isolate the needle formation 1 housed within the needle holder 40 in an aseptic condition. The open end can be opened by removing the seal member 44 from the flange 42 using a finger or the like.

Next, an operation method of a percutaneous medication device 5 of the present invention will be described.

Since the needle formation 1 is stored in the needle holder 40 in an aseptic condition, the seal member 44 of the needle holder 40 is removed from the flange 42 (FIG. 2).

Next, the Luer lock portion 20 formed at the tip of the syringe 6 is engaged with the flange 12 of the needle formation 1 housed within the needle holder 40, and the syringe 6 is rotated in this state. Since the needle formation 1 cannot rotate relative to the needle holder 40, the Luer lock portion 20 of the syringe 6 and the flange 12 of the needle formation 1 are engaged with each other. In this state, as the syringe 6 is pulled out from the needle holder 40, the needle formation 1 is attached to the tip of the syringe 6 (FIG. 1).

Then, in accordance with an ordinary method, the plunger S of the syringe 6 is pressed for operation, and a medical liquid in the syringe 6 enters a space portion 36 of the needle formation 1, and further, the medical liquid is pushed out from the discharge opening 13 of the needle 11 through each needle 11 of the needle formation 1.

In the present invention, a plurality of needles 11 protrude from the surface of the needle formation 1, the respective discharge openings 13 formed at the tips of the needles 11 face outwards, and the intervals (pitches) of adjacent needles 11 is 1 mm to 10 mm (1 mm to 5 mm or 5 mm to 10 mm). Therefore, the medical liquid can be injected into a desired part of the skin.

The medial liquid used in the percutaneous medication device 5 is, typically, a solution, gel or suspension, which contains a medical agent. The usable medical agent is not substantially limited, except for the medical agents that are not suitable for percutaneous administration.

The following are examples of main medical agents used in the present invention: Hyaluronic acid, collagen, botox, antimicrobial agents, virucide, vaccine, antitumor agents, immunosuppressant, steroid, antiphlogistic, antirheumatic, antiarthritic, antihistamines, antiallergic agents, diabetes drugs, hormonal agents, bone/calcium metabolic agents, vitamins, hematic preparation, hematinic, antithrombotic agents, antihyperlipidemia agents, antiarrhythmic agents, vasodilator, prostaglandin, calcium antagonist, ACE inhibitor, beta blocker, depressor, diuretic, xanthine derivative, beta agonist, antiasthma agents, antitussive, expectorant, anticholinergic agents, stegnotic, stomachic digestant, antiulcerative, cathartic, narcoleptic, sedative, antipyretic, cold medicine, antiepileptic agents, antipsychotic agents, antidepressant, antianxiety agents, central nervous system stimulant, parasympathomimetic agents, sympathetic agents, antiemetic, analeptic, antiparkinsonian agents, muscle relaxant, antispasmodic, anesthetic, antipruritic agents, antimigraine headache agents, diagnostic agents, oligonucleotide, gene agents and the like.

Here, the medical agent is preferably protein, peptide, polysaccharide, oligonucleotide, DNA or the like, which do not express the effect or diminish in a peroral administration. More particularly, the medical agent is high molecular weight drag such as insulin, growth hormone, interferon, calcitonin, and the like.

While the discharge openings 13 formed at the tips of the respective needles 11 are formed to face the direction substantially opposite from the center direction of the needle formation 1 in the embodiment above, the discharge openings 13 formed at the tips of the respective needles 11 may be formed to face substantially the center direction of the needle formation 1 as shown in FIG. 15. In this case, the discharge opening 13 being substantially inwards means that the discharge opening 13 is facing the center direction of the needle formation 1 and, from the view point of the axial direction, the deviation angle theta2 of a segment L2 connecting the upper end edge and the lower end edge of the discharge opening 13 is within 15 degrees with respect to a segment L1 connecting the central axis of the needle 11 and the center (P) of the needle formation 1.

The cross-sectional shape of the needle formation 1 need not be circular, but may be a square, an ellipse or an oval shape. Furthermore, while the recess groove (spline) is provided in the inner surface of the wall of the needle holder 40 to be engaged with the protruding portion formed on the outer surface of the needle formation 1, a recess with a cross sectional shape of a triangle, a hexagon, a square, an ellipse, an oval or the like may be formed on the inner side of the needle holder 40, and the head portion 25 of the cover portion 24 may be in a shape to engage with the recess, so that the recess will engage with the head portion 25 of the cover portion 24, being unable to rotate relative to the head portion 25. Further, a taper may be provided for the recess 43 of the needle holder 40 so that the tip of the needle formation 1 will be configured to be readily inserted into the recess 43.

While three of the needles 11 are formed at the tip of the needle formation 1 away from the center with the same distance and at the positions away from each other at 120 degrees, it will not limit the arrangement and the number of the needles 13.

### INDUSTRIAL APPLICABILITY

The use of the percutaneous medication device according to the present invention allows percutaneous administration of a medical agent, a cosmetic, or the like at a desired position of the skin, and the use does not involve pain. Further, the percutaneous medication device according to the present invention allows a medical liquid to be injected and dispersed at a predetermined tissue layer in an effective manner, so that the dose of the medical liquid can be restrained to be small, and the medical liquid can be minimally invasive.

### REFERENCE SIGNS LIST

1 needle formation

5 percutaneous medication device

6 syringe

10 needle formation body

11 needle

13 discharge opening

18 fixation member

24 cover portion

30 space

40 needle holder

1. A percutaneous medication device comprising: a syringe into which a medical liquid is filled; and a needle formation attached to a tip of the syringe; wherein:
the needle formation comprises a plurality of needles protruding from a surface of a tip of the needle formation; a tip of the needle is cut diagonally with respect to the longitudinal direction of the needle; a
discharge opening formed at the tip of each needle is facing substantially opposite from the center direction of the needle formation;
and a pitch between the adjacent needles is 1 mm to 10 mm.

2. The percutaneous medication device according to claim 1, wherein the number of needles protruding front the surface of the tip of the needle formation is 2 to 10.

3. The percutaneous medication device according to claim 2, wherein the number of needles protruding from, the surface of the tip of the needle formation is three.

4. The percutaneous medication device according to claim 1, wherein the protruding dimension of the needle protruding from the surface of the tip of the needle formation is within the range of 0.1 mm to 10 mm.

5. The percutaneous medication device according to claim 1, wherein a pitch between the adjacent needles is within the range, of 1 mm to 3 mm.

6. The percutaneous medication device according to claim 1, wherein:
the needle formation comprises a needle formation body, in which the needle protrudes from the surface of the tip thereof, and a cover portion for covering a tip portion of the needle formation body;
the needle is inserted into a through-hole formed at the tip portion of the needle formation body and the needle is fixed to the needle formation body by a fixation member;
the needle formation body is engaged with the covering portion, and a space is formed between the inner surface of the covering portion and the tip surface of the needle formation body; and
the needle protrudes from a surface, of a tip of the covering portion, through a needle insertion hole formed at a tip portion of the covering portion.

7. A needle formation attached to a tip portion of a syringe, wherein:
the needle formation comprises a plurality of needles protruding from, a surface of a tip of the needle formation;
a tip of each of the needles is cut diagonally with respect to the longitudinal direction of the needle;
a discharge opening formed at the tip of each of the needles is facing substantially opposite from the center direction of the needle formation; and
a pitch between the adjacent needles is within the range of 1 mm to 10 mm.

8. A percutaneous medication device comprising: a syringe into which a medical liquid is filled; and a needle formation attached to a tip of the syringe; wherein:
the needle formation comprises a plurality of needles protruding from a surface of a tip of the needle formation;
a tip of each of tire needles is cut diagonally with respect to the longitudinal direction of the needle;
a discharge, opening formed at the tip of each of the needles is facing substantially the center direction of the needle formation; and
a pitch between the adjacent needles is within the range of 1 mm to 10 mm.

9. A needle formation attached to a tip portion of a syringe, wherein:
the needle formation comprises a plurality of needles protruding from a surface of a tip of the needle formation;
a tip of each of the needles is cut diagonally with respect to the longitudinal direction of the needle;
a discharge opening formed at the tip of each of the needles is facing substantially the center direction of the needle formation; and
a pitch between the adjacent needles is within the range of 1 mm to 10 mm.
